(19) United States
(12) Patent Application Publication
(10) Pub. No.: US 2017/0325999 A1
(43) Pub. Date: Nov. 16, 2017

(54) EYE DROP BOTTLE WITH ALIGNMENT INDENTATION

(71) Applicant: BEDO SOLUTIONS, LLC, Portland, OR (US)

(72) Inventor: Robert Kinast, Portland, OR (US)

(21) Appl. No.: 15/666,511

(22) Filed: Aug. 1, 2017

Related U.S. Application Data
(63) Continuation-in-part of application No. PCT/US2016/044328, filed on Jul. 27, 2016.
(60) Provisional application No. 62/199,795, filed on Jul. 31, 2015.

(51) Int. Cl. A61F 9/00

(52) U.S. Cl. CPC A61F 9/0026

(57) ABSTRACT

An improved eye drop bottle (10) that improves adherence in the dispensing of eye drops to is claimed. The bottle includes an indentation (45) in the eye drop bottle to facilitate better administration of eye drops. The indentation (45) defines an engagement surface (55) of the bottle (10). The engagement surface (55) straddles the bridge of a user's nose (80), substantially supporting the eye drop bottle (10), such that the reservoir (20) of the bottle (10) does not visually intrude upon the temporal half of the field of view of the user's eye or approach the eye directly along the forward line of sight of the user's eye.

### CROSS REFERENCE TO RELATED APPLICATION

This application is a Continuation-in-Part of International Application No. PCT/US2016/044328 filed Jul. 27, 2016 entitled “Eye Drop Dispensement Aid,” which claims the benefit under 35 U.S.C. §119(e) of U.S. Provisional Application Ser. No. 62/199,795, filed on 31 Jul. 2015 and of U.S. Provisional Application Ser. No. 62/287,874, filed on 27 Jan. 2016, the contents of which are incorporated herein by reference in their entirety.

### FIELD OF THE INVENTION

The present invention relates, generally, to devices and methods for delivery of medication and improving the rate of successful medication adherence. More particularly, the present invention relates to improving the rate of successful delivery of medication in the form of eye drops from an eye drop bottle to an eye.

### BACKGROUND

A number of conditions, including glaucoma, conjunctivitis and allergies may be treated by the use of eye drops or an eye drop spray, aka eye spray. Individual eye drops may contain steroids, antihistamines, antibiotics, prostaglandin analogs, beta blockers, alpha agonists, carbonic anhydrase inhibitors, or other drugs. Additionally, eye drops or an eye spray may be used to lubricate the eye or replace tears. In order for the treatment with eye drops or an eye spray to be effective, good adherence to treatment, i.e., remembering to use the eye drop and getting sufficient amount of the eye drop liquid (whether in drop or spray form) into the eye, is needed. Some adherence studies of glaucoma patients indicate that approximately 45% of patients demonstrate poor ability to remember to use their eye drops, and other studies show that when patients do attempt to administer eye drops, they frequently miss the eye.

One reason for poor adherence is the inability to successfully administer an eye drop or spray. Patients may accidentally miss the eye instead placing the liquid on the eyelids or cheek, or they may touch the bottle tip against the ocular surface causing contamination of the bottle and possible ocular surface damage. In a recent study, over 30% of patients missed the eye while attempting to instill an eye drop and over 75% touched the bottle tip to the ocular surface or eyelids. Only 10% of patients exhibited proper technique. See e.g., Gupta R, Patil B, Shaw B M, “Evaluating eye drop instillation technique in glaucoma patients,” Journal of Glaucoma, 2012, 21: 189-92. Correctly placing an eye drop through conventional means is challenging.

Researchers and eye care providers have had difficulty developing a suitable device to improve the success rate of instilling eye drops. Existing delivery devices have not been widely adopted due to their difficulty of use, intimidating nature, and lack of efficacy. Scholarly articles have examined many of the options, including “Aids for Eye Drop Administration,” Isiah Davies, et al., Survey of Ophthalmology, Vol. 62, No. 3, pp. 332-345, 2017; “Evaluation of the efficacy and safety of a new device for eye drops instillation in patients with glaucoma,” Daniela Junqueira, et al., Clinical Ophthalmology, Vol. 2015:9, pp. 367-371, 2015; “Xal-Ease®: impact of an ocular hypotensive device on ease of eyedrop administration, patient compliance, and satisfaction,” Jean-Philippe Nordmann, et al., European Journal of Ophthalmology Vol. 19, No. 6, pp. 949-956, 2009; and “Evaluation of an eye drop guide to aid self-administration by patients experience with topical use of glaucoma medication,” Asif Alyani, et al., Canadian Journal of Ophthalmology, Vol. 40, No. 2, pp. 170-174, 2005.

Unsuccessful attempts to address these problems which may be examples of prior art are: U.S. Published Patent Appl'ns Nos. US2010-160872, US2010-174248, US2010-286634, US2012-150132, and US2014-371688; U.S. Pat. Nos. 2,482,431; 2,676,592; 2,898,911; 3,446,209; 3,521,636; 3,872,865; 4,085,750; 4,471,890; 4,531,944; 4,733,802; 4,834,727; 4,960,407; 5,059,188; 5,569,224; 5,578,019; 5,836,911; 5,902,292; 6,010,488; 6,149,625; 6,258,059; 6,595,970; 6,632,202; 7,235,065; 7,527,613; 8,206,362; 8,216,195; 8,734,408; 8,348,912; 9,033,941; 9,072,581; and 9,486,356; European Patent Publ'ns EP0013187A1 and E00934921; and WIPO Publ'ns WO/1996/040025A1, and WO/1997/027834A1. Accordingly, there is a need in the art for an improved eye drop bottle with more successful, easier, and less intimidating eye drop delivery.

A successful device for instilling eye drops or a spray would incorporate a reservoir for holding the eye drop or spray liquid and have a surface that fits firmly against the upper nose/bridge of the nose/nasion area avoiding contact with the eyelids and orbital rim and periocular tissues and eyebrow, and the bottle or reservoir would be fully supported across the bridge of the nose and align the bottle downward towards the ocular surface so that the bottle tip would be above the surface without touching it, permitting the dispensing tip to hover over the eye. The bottle should avoid having any substantial portion of the bottle, other than the dispensing tip itself, crossing over the eye to intrude upon the temporal field of view of the eye (the half of the field of view extending outward from the body center-line). The structure of the bottle would prevent lateral motion of the bottle and stabilize the reservoir to aid in the administration of eye drops or an eye spray.

Additionally, a successful device for improving adherence could also include the ability to monitor adherence and alert when medication is due, through the inclusion of electronics for this function.

### BRIEF DESCRIPTION OF THE DRAWINGS

The features, aspects and advantages of the present invention will become better understood with regard to the following description, appended claims and accompanying figures wherein:

FIG. 1 illustrates an embodiment of an eye drop/eye drop spray bottle.

FIG. 2 illustrates use of an eye drop/eye drop spray bottle from FIG. 1.

FIG. 3 illustrates an alternative embodiment of an eye drop/eye drop spray bottle with an alignment indentation.

FIG. 4 illustrates an alternative embodiment of an eye drop/eye drop spray bottle with an alignment indentation.

FIG. 5 illustrates an alternative embodiment of an eye drop/eye drop spray bottle with an alignment indentation.

### DETAILED DESCRIPTION

In the following description of the preferred embodiments, reference is made to the accompanying drawings which show by way of illustration specific embodiments in which the invention may be practiced. Wherever possible, the same reference numbers will be used throughout the drawings to refer to the same or like parts. It is to be understood that other embodiments may be utilized and structural and functional changes may be made without departing from the scope of the present invention. It is to be understood that as used herein, the directional term medial means toward the line of symmetry of the human body and the directional term lateral means away from the line of symmetry of the human body. Similarly, when referring to the field of view of an eye, the nasal field of view extends medially from a vertical plane aligned with the directly forward-looking line of sight of the eye and the temporal field of view extends laterally from the same vertical plane. It is also to be understood that unless specifically differentiated, the features of the embodiments may be used in bottles which dispense eye drops in drop form and bottles which dispense eye drops in spray form and the term eye drop bottle may refer to a bottle which dispenses eye drops in spray form.

A number of conditions, including glaucoma, allergies, and dryness may be treated by the use of eye drops. Eye drops may contain steroids, antihistamines, antibiotics. prostaglandin analogs, beta blockers, alpha agonists, carbonic anhydrase inhibitors, or other drugs. Additionally, eye drops or sprays may be used to lubricate the eye or replace tears. In order for the treatment with eye drops or sprays to be effective, good adherence to treatment, i.e., remembering to use the eye drops or sprays and getting sufficient amount of the medicine into the eye, is needed.

Approximately 45% of glaucoma patients demonstrate poor ability to remember to use their eye drops (defined as using less than 75% of expected eye drop doses) and about 30% of glaucoma patients miss the eye while administering eye drops. Poor adherence increases the risk of blindness in patients with glaucoma. In addition to glaucoma treatment, medication adherence affects all eye diseases treated with topical medications, whether antibiotics for a corneal ulcer or steroids for uveitis.

Successfully administering eye drops or sprays presents many challenges including dexterity difficulties, complex spatial orientation, and patient fear. Successfully administering an eye drop requires that the bottle tip remain in precise position over the globe without contacting the ocular surface while applying force to the bottle. This complex task is a challenge for all patients, especially the elderly and those with a tremor or arthritis. Patients are often fearful of contact with the eyes, which can make drop instillation more difficult.

Because of these issues, researchers and eye care providers have had difficulty developing a suitable device to improve the success rate of instilling eye drops. Prior devices have included large, clunky, awkward, and/or unstable aspects that fit over the entire globe or touch the eyelids or orbital rim or eyebrow or are prone to movement around the eye. Devices that touch periocular tissues and are visible over the eye can intimidate patients and limit adoption.

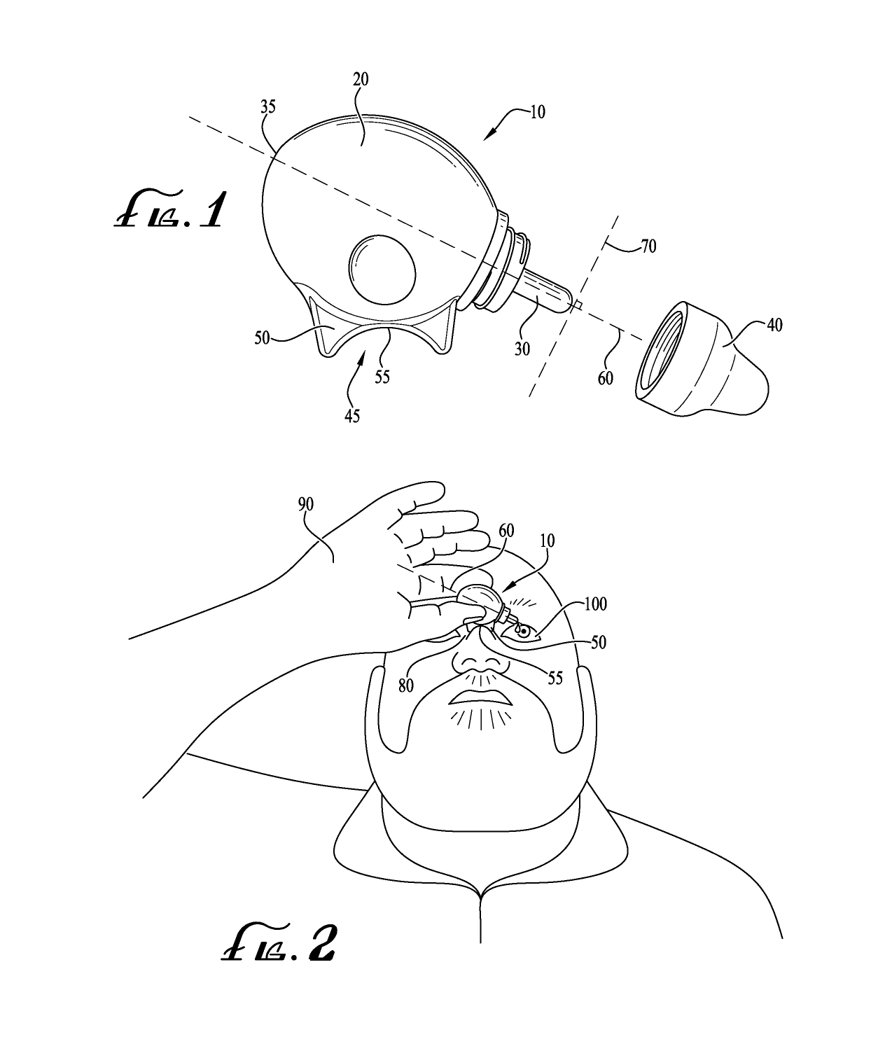

The invention provides for a secure, stable, easily manipulated, eye drop delivery device which does not block or impede upon the temporal field-of-view of the user's eye, when the user is applying eye drops or an eye spray. FIG. 1 illustrates an embodiment of an eye drop bottle 10 according to the invention. The bottle comprises a reservoir 20, a tip 30 through which the eye drops or spray are delivered, a cap 40, that allows the bottle to be securely closed, and an alignment structure 50, which includes an engagement surface 55 that may take the form of an anchor or footplate. The engagement surface 55 is formed by an arcuate indentation 45 into the alignment structure such that the engagement surface 55 engages both sides of the bridge of the user's nose 80. As used herein throughout “bridge” refers to the general area of the upper or mid-nose and lower forehead proximate to the nasal bones and glabella area. For purposes of this description, the primary axis 60 of the bottle is an axis extending through the center of the tip 30, perpendicular to the face 70 of the tip 30 and extending through the base 35 of the bottle. It would also be obvious to a person of ordinary skill in the art that the reservoir 20 and tip 30 and alignment 50 structure could take the form of various dimensions, shapes, sizes, and configurations; for example, two tips could be included on opposing sides of the reservoir 20 to allow simultaneous bilateral eye drop delivery.

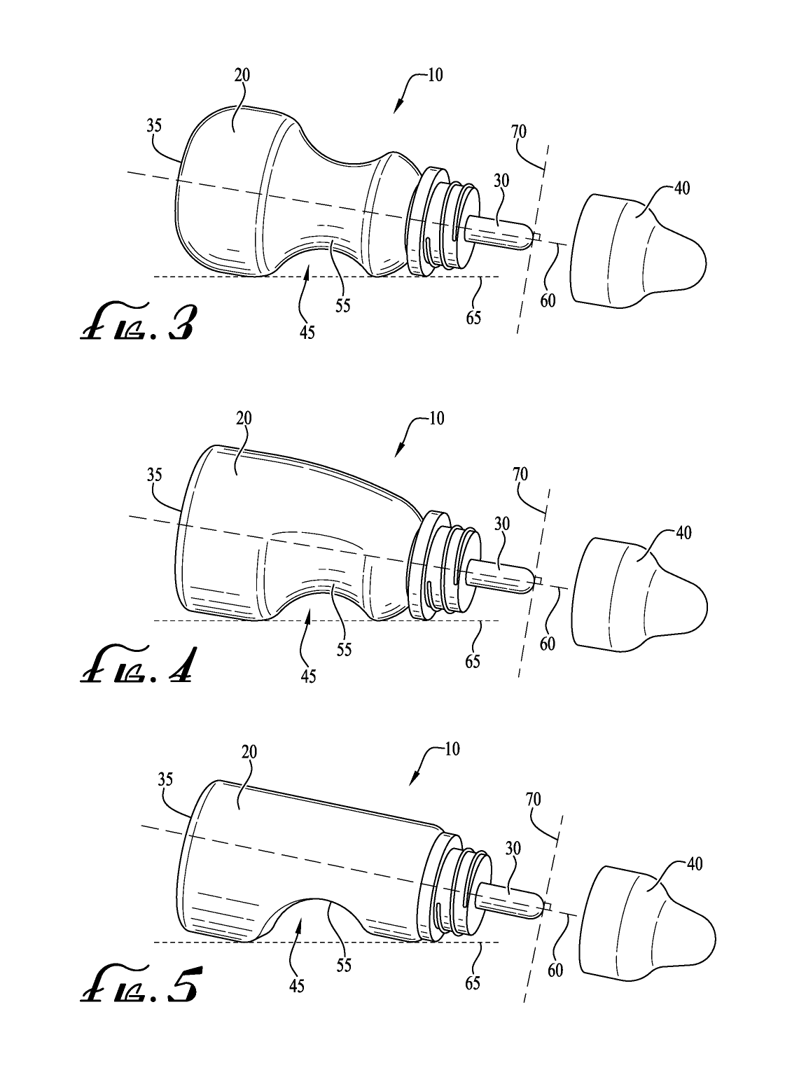

FIGS. 3-5 illustrate alternative embodiments of the invention in which the arcuate indentation 45 that forms the engagement surface 55 is formed directly into the bottle without a dedicated alignment structure.

FIG. 3 illustrates a further embodiment of the invention in which the indentation 45 that forms the engagement surface 55 is formed directly into the reservoir 20 of the bottle 10. The engagement surface 55 may optionally be textured to improve the grip with the bridge of the user's nose. In FIG. 3, the apices of the curves which form the indentation 45 that forms the engagement surface 55 form a second arc which fully encircles the primary axis 60 of the bottle 10. It would be obvious to one of skill in the art that the circuate arc formed by the apices of the indentation 45 may be centered on the primary axis 60 of the bottle or be located off-center from the primary axis 60 of the bottle 10 and that by choice of the form of circuate arc and the cross-section of the bottle at the apices of the indentation 45 the depth of the indentation 45 into the bottle 10 may be varied about the circumference of the bottle 10. Thus the relative placement of the indentation 45 in the reservoir 20 may be adjusted to allow for a preferred alignment of the bottle 10. Said relative placement can preferentially be achieved such that the natural level line 65 achieved when placing the bottle 10 over the bridge of the nose 80 results in natural tilt of the primary axis 60 of the bottle that facilitates flow of eye from the reservoir 20 to the tip 30.

FIG. 4 illustrates a further embodiment of the invention in which the arcuate indentation 45 that forms the engagement surface 55 is formed directly into reservoir 20 of the bottle 10. In this embodiment, the apices of the curves which form the indentation 45 form a second arc which partially encircles the bottle 10. This provides for greater freedom to rotate the bottle 10 about its primary axis 60, while continuing to have the engagement surface 55 rest on the bridge of the user's nose 80 providing support to and precluding lateral movement of the bottle 10. The engagement surface 55 may be textured so as to improve the grip on the bridge of the user's nose. As with FIG. 3, a preferred alignment of the bottle can preferentially be achieved such that the natural level line 65 achieved when placing the bottle 10 over the bridge of the nose 80 results in natural tilt of the primary axis 60 of the bottle that facilitates flow of eye from the reservoir 20 to the tip 30.

FIG. 5 illustrates an embodiment of the bottle 10 with a basic cylindrical shape including an arcuate indentation 45 directly into the side of the reservoir 20 of the bottle 10 to form an engagement surface 55. In FIG. 5, the apices of the curves which form the indention 45 as it is cut through the bottle form a line which is perpendicular to the primary axis 60 of the bottle 10. A preferred alignment of the bottle can preferentially be achieved such that the natural level line 65 achieved when placing the bottle 10 over the bridge of the nose 80 results in natural tilt of the primary axis 60 of the bottle that facilitates flow of eye from the reservoir 20 to the tip 30.

The eye drop bottle in question would also be able to be manufactured at low cost and in substantial numbers and be able to utilize modern mass production techniques such as blow molding, including extrusion and injection molding. Inexpensive, light weight plastics are preferred for the mass manufacture of such eye drop bottles, have been approved by regulatory authorities for use in eye drop bottles. The nature of the present invention is particularly well suited to the use of such production techniques and materials while maintaining the advantages of the invention.

Although the figures show embodiments in which the cross-section of the reservoir about the primary axis of the bottle is a smooth closed curve, such as a circle or ellipse, it would be obvious to one of ordinary skill in the art that modern molding techniques including injection or blow molding will allow for other closed forms, including irregular shapes. In this manner, the bottle may be given shapes which better engage with the user's hand or are readily identifiable by appearance or feel.

The use of an embodiment of the bottle 10 is illustrated in FIG. 2. The engagement surface 55 of the bottle 10 is securely placed against the bridge of the user's nose 80 and may optionally be textured to provide for improved grip against the bridge of the user's nose. The tip 30 of the bottle 10 is then aligned by the engagement surface 55 into position over the user's eye 100. Lateral movement of the bottle is controlled by an indentation 45 into the alignment structure 50. The indention 45 in the alignment structure 50 allows the engagement surface 55 to straddle the bridge of the user's nose 80, limiting lateral movement, aligning the bottle 10, and limiting movement of the bottle 10 during application of the eye drops. Further the engagement surface 55 requires sufficient width or adequate shape to provide a stable position on the bridge of the user's nose 80 for the apex of the indentation 45, such that the tip of the bottle does not unintentionally move vertically relative to the user's eye 100. The bottle rests on the bridge of the user's nose 80 and is stabilized by the user's hand 90, which may also squeeze the bottle 10 to administer the eye drops through the tip 30. Alternatively, the bottle may contain a spray delivery mechanism in which a button or cap is pressed to cause a spray or mist of eye drop liquid to be introduced to the user's eye. Because the primary axis 60 of the bottle 10 is rotated medially from the forward line of sight of the user about the apex of the indentation 45 of the alignment structure 50, the bottle 10 does not approach the eye directly along, or parallel to, the forward line of sight of the user's eye 100. Thus, neither the user's hand 90 nor the body of the bottle 10 (i.e., the portion of the bottle 10 excluding the tip 30), extend into the temporal half of the field of view of the user's eye 100, and the application method incites less consternation and anxiety in users, as users are accustomed, when focusing closely, to have an obstruction in the nasal half of their field of view, i.e., their nose, but are not typically accustomed to obstructions directly in their forward line of sight or in the temporal half of their field of view. The orientation of the primary axis 60 of the bottle 10 results in raising the base 35 of the bottle 10 and improving flow of eye drop liquid to the tip 30 of the bottle 10. The compact and simple shape of the bottle 10 also alternatively permits traditional free-hand direct eye drop administration.

Fear or anxiety regarding the application of eye drops or sprays can result in lack of cooperation in the application of eye drops and directly affect the efficacy of treatment, especially in children. See Sujuan, Jane Lim, et al., “The psychological impact of eye drops administration in children,” Journal of the AAPOS, Vol. 19, No. 4, August 2015, pp. 338-343. Decreasing or ameliorating this anxiety by avoiding intrusions into the temporal half of the field of view or along the forward line of sight of the user's eye provides significant benefit.

In the United States, the Food and Drug Administration regulates packaging for medical eye drops. See, e.g., “Container Closure Systems for Packaging Human Drugs and Biologics,” Chemistry Manufacturing, and Controls Documentation, U.S. Dept. of Health and Human Services, Food and Drug Administration, May 1999. The present invention, when embodied in an eye drop bottle would be able to meet any suitability requirements, including those relating to protection, safety, compatibility, and performance, as well as quality control and stability requirements. Additionally, any bottle would need to be approved by the FDA for the specific medication it will contain because of concerns regarding sterility, drop quantity, and drop volume. It would be clear to a person of ordinary skill in the art that specific changes to the materials used in the manufacture, adoption of specific dimensions and features for the tip and any cap affixed to the tip for sealing the bottle can be made in the implementation of a bottle in accord with the invention.

A further feature of the present invention is the ability to incorporate an adherence auditing mechanism which can detect when the ophthalmic eye drop are being administered, and optionally, in what amount. This mechanism can take the form of electronics which is contained within the invention, optionally, within the alignment structure 50, which can detect when an eye drop is being administered, track and share adherence data, and alert when an eye drop medication is due.

Current eye drop bottles do not include the physical space or intrinsic capacity for an adherence monitoring mechanism. The alignment structure 50 permits room for electronics in the bottle 10, and the adjacent positioning of the eye drop bottle tip 30 to the electronics 50 can permit a mechanical or non-mechanical counting mechanism while still permitting a watertight FDA approved bottle closure.

It is would be obvious to one of ordinary skill in the art that there are numerous ways to identify and detect administration. These include monitoring, via optical, mechanical, electronic or magnetic sensors, when the cap 40 is removed from and/or attached to the bottle 10, or when an eye drop or spray exits the bottle tip 30 or passes by a sensor. This may also include sensors to detect when the bottle 10 or pre-existing multi-use eye drop container is squeezed to dispense eye drops or sprays. Accuracy of such detection may be improved by position sensors which can confirm the orientation of the bottle or pre-exiting ophthalmic eye drop container. The adherence auditing mechanism may also include recording the date, time, and duration, of use and may include communications capabilities such as through Wi-Fi, Bluetooth, or other wireless communications protocols to communicate the monitoring data to other devices.

There is disclosed in the above description and the drawings, an eye drop bottle with an alignment structure that fully and effectively overcomes the disadvantages associated with the prior art. However, it will be apparent that variations and modifications of the disclosed embodiments may be made without departing from the principles of the invention. The presentation of the preferred embodiments herein is offered by way of example only and not limitation, with a true scope and spirit of the invention being indicated by the following claims.

Any element in a claim that does not explicitly state “means” for performing a specified function or “step” for performing a specified function, should not be interpreted as a “means” or “step” clause as specified in 35 U.S.C. §112.

What is claimed is:

1. An eye drop bottle comprising:
an opening for dispensing eye drop liquid;
a reservoir for holding eye drop liquid;
an indentation into the eye drop bottle forming an engagement surface;
wherein, when positioned to apply eye drop liquid to a user's eye, the bottle is substantially supported upon the bridge of the user's nose;
wherein the engagement surface straddles the bridge of the user's nose; and
wherein the reservoir of the eye drop bottle does not extend into the temporal half of the field-of-view of the user's eye and does not approach the eye directly along the forward line of sight of the user's eye, when positioned to apply eye drops or an eye spray to the user's eye.

2. The eye drop bottle of claim 1, wherein the primary axis of the eye drop bottle, when the eye drop bottle is positioned to apply eye drop liquid to a user's eye with the bottle substantially supported upon the bridge of the user's nose and the engagement surface straddling the bridge of the user's nose, is tilted so as to allow gravity to assist the flow of the eye drop liquid from the reservoir to the opening for dispensing eye drops.

3. The eye drop bottle of claim 1, wherein the opening for dispensing eye drop liquid dispenses the eye drop liquid in drop form.

4. The eye drop bottle of claim 1, wherein the opening for dispensing eye drop liquid dispenses the eye drop liquid in spray or mist form.

5. The eye drop bottle of claim 1 further comprising:
an alignment structure integrated to the reservoir,
wherein the indentation into the eye drop bottle forming an engagement surface is formed in the alignment structure structurally integrated to the bottle.

6. The eye drop bottle of claim 1, wherein the reservoir is predominately cylindrical in shape, wherein the indentation is arcuate in shape, and a line formed by the apices of the indentation is perpendicular to the primary axis of the cylindrical shape of the reservoir.

7. The eye drop bottle of claim 1, wherein the cross section of the reservoir is predominately circular or elliptical in shape around the primary axis of the reservoir, wherein the indentation is arcuate in shape, and the apices of the indentation form a second arcuate curve which partially encompasses the circumference of the reservoir.

8. The eye drop bottle of claim 7, wherein the indentation extends less than 50% of the way around the circumference of the reservoir.

9. The eye drop bottle of claim 7, wherein the indentation extends between 50% and 75% of the way around the circumference of the reservoir.

10. The eye drop bottle of claim 1, wherein the cross section of the reservoir is predominately circular or elliptical in shape around the primary axis of the reservoir, wherein the indentation is arcuate in shape, and the apices of the indentation form a second arcuate curve which fully encompasses the circumference of the reservoir.

11. The eye drop bottle of claim 10, wherein the second arcuate curve formed by the apices of the indentation is the same shape as the cross section of the reservoir around the primary axis of the reservoir at the apex of the indentation.

12. The eye drop bottle of claim 11, where in the center of the second arcuate curve formed by the apices of the indentation is centered on the primary axis of the eye drop bottle.

13. The eye drop bottle of claim 1, wherein the cross section of the reservoir is a closed smooth curve around the primary axis of the reservoir, wherein the closed smooth curve is neither circular or elliptical, wherein the indentation is arcuate in shape, and the apices of the indentation form a second arcuate curve which partially encompasses the circumference of the reservoir.

14. The eye drop bottle of claim 1, wherein the cross section of the reservoir is a closed smooth curve around the primary axis of the reservoir, wherein the closed smooth curve is neither circular or elliptical, wherein the indentation is arcuate in shape, and the apices of the indentation form a second arcuate curve which fully encompasses the circumference of the reservoir.

15. The eye drop bottle of claim 1, wherein the cross section of the reservoir is an irregular closed curve around the primary axis of the reservoir, wherein the indentation is arcuate in shape, and the apices of the indentation form a second arcuate curve which partially encompasses the circumference of the reservoir.

16. The eye drop bottle of claim 1, wherein the cross section of the reservoir is an irregular closed curve around the primary axis of the reservoir, wherein the indentation is arcuate in shape, and the apices of the indentation form a second arcuate curve which fully encompasses the circumference of the reservoir.

17. A method for dispensing eye drops from an eye drop bottle comprising the steps of:
placing an engagement surface of the bottle to straddle the bridge of a user's nose;
aligning the eye drop bottle about the engagement surface to align an opening of the eye drop bottle in a desired position relative to a user's eye;
manually engaging the eye drop bottle to cause some amount of an eye drop liquid to exit from the eye drop bottle via the opening in the eye drop bottle and into the user's eye without the eye drop liquid making further contact with the bottle;
wherein the engagement surface is formed by an indentation into the eye drop bottle;
wherein the weight of the eye drop bottle and its contents are predominately supported upon the bridge of the user's nose;
wherein the only contact between the bottle and the user's face is between the engagement surface and the bridge of the user's nose; and
wherein the body of the eye drop bottle does not extend into the temporal half of the field-of-view of the user's eye and does not approach the eye directly along the forward line of sight of the user's eye, when positioned to apply eye drops or an eye spray to the user's eye.

18. The method of claim 17 wherein the step of manually engaging the eye drop bottle comprises squeezing the eye drop bottle to cause the eye drop liquid to exit from the opening in the eye drop bottle in drop form.

19. The method of claim 17 wherein the step of manually engaging the eye drop bottle comprises pressing an actuator in the eye drop bottle to cause the eye drop liquid to exit from the opening in the eye drop bottle in spray form.
